(19) United States
(12) Patent Application Publication
(10) Pub. No.: US 2004/0191326 A1
(43) Pub. Date: Sep. 30, 2004

(54) Taste-masking vehicle for coated oxazolidinone particles

(76) Inventors: Joseph P. Reo, Kalamazoo, MI (US); Jennifer K. Fredrickson, Kalamazoo, MI (US); Julia Kistka, Kalamazoo, MI (US)

Correspondence Address: WARNER-LAMBERT COMPANY, 2800 PLYMOUTH RD, ANN ARBOR, MI, 48105, US

(21) Appl. No.: 10/790,312

(22) Filed: Mar. 1, 2004

Related U.S. Application Data
(60) Provisional application No. 60/459,382, filed on Mar. 31, 2003.

(51) Int. Cl. A61K 31/5377; A61K 31/4439; A61K 31/421; A61K 9/16; A61K 9/50

(52) U.S. Cl. 424/490; 514/235.2; 514/340; 514/376

(57) ABSTRACT

The present invention is directed to dry formulations of coated oxazolidinone particles, such as coated linezolid particles, which, when suspended in an aqueous solution, mask the taste of the oxazolidinone in the resulting suspension vehicle. Specifically, a mixture of sugars in the formulation, comprising sorbitol and sucrose, inhibit mass transport of the oxazolidinone from the particles into the suspension vehicle. Dry formulations of the coated oxazolidinone particles, suspensions of the same, and methods of using the same to treat or prevent infections from gram positive bacteria are all disclosed.

### CROSS REFERENCE TO RELATED APPLICATIONS

The present patent application claims the benefit of U.S. provisional patent application serial No. 60/459,382, filed Mar. 31, 2003.

### FIELD OF THE INVETION

The present invention relates to suspension vehicles that mask objectionable taste of at least one drug, such as an oxazolidinone. The present invention, specifically, relates to suspension vehicles that mask such objectionable taste by limiting the transport of drug from micron sized drug particles coated with a polymer film, such as microencapsulated oxazolidinone particles or coacervated oxazolidinone particles.

### BACKGROUND

Objectionable taste is generally not a significant concern in the oral administration of solid dosage forms. Such dosage forms, such as capsules, caplets, or tablets are usually intended to be swallowed whole. To the extent any making is necessary, the objectionable taste of any active agent in a solid dosage form can be masked by an exterior coating.

However, children, older persons, adults with small esophageal passages, and many other persons including disabled or incapacitated subjects often have trouble swallowing solid dosage forms. In such situations, it is desirable to be able to provide the active agent(s) in the form of a liquid dosage form. A liquid dosage is particularly preferred because of the ease with which it can be administered and ingested. Liquid dosages are also preferred because of the increased likeliness of compliance by subjects in taking such dosage forms, particularly for subjects for whom it is difficult or even impossible to take solid dosage forms of the same active agent(s).

Unlike solid dosage forms, the objectionable taste of an active agent in a liquid dosage form is generally considerably more difficult to mask. The disagreeable taste of some active agents can be masked by the addition of natural or artificial sweeteners. However, such sweeteners alone are insufficient to mask the objectionable taste of some agents. Microencapsulation of active agent(s) has also been used to mask taste. However, as noted in U.S. Pat. No. 5,633,006 (Cantania et al., col. 2, lines 15-18) microencapsulation, alone, of potently bitter active agents, such as azithromycin, provides insufficient taste masking.

Various suspension vehicles have been developed for use in masking the taste of microencapsulated active agents. U.S. Pat. No. 6,197,348 (Morella et al.) discloses that taste-masking in an aqueous suspension of microencapsulated active agent is a function of the properties of the polymer coating of the microcapsules and the pH of the suspension vehicle. In that case, the pH of the suspension vehicle used is one in which the microencapsulated active agent is poorly soluble.

EP 0 717 992 A2 (McNEIL-PPC, INC. for an invention by Ratnaraj et al.) discloses a controlled release powder including microencapsulated acetaminophen, comprising cellulose acetate butyrate or ethylcellulose in a polymer matrix. A suspension of the controlled release powder was also disclosed. Several different sugars were listed as possible sweetening agents that could be added to the suspension. However, no suggestion was made that a mixture of any two or more sugars would be suitable for use in sweetening or in taste-masking.

U.S. Pat. No. 4,994,260 (K ällstrand et al.) discloses a release controlling substance (referred to as a “sink”) that controls the release of a drug from microcapsules of the drug. The sink was described therein as suitably including a carbohydrate or a carbohydrate-related compound, such as a poly- or oligo-saccharide, a disaccharide, or a monosaccharide, or a mixture of two or more of the same. Examples of sugars cited as suitable for use in the sink included sucrose, glucose, fructose, and sorbitol. The amount of sink is described as being “between 40% and 99% (weight/weight), preferably 60-75% (weight/weight) of the entire preparation, that is of the ready to use suspension for oral administration.” ('260 patent, col. 2, lines 34-37). The Examples section illustrates the use of sinks of various individual sugars or mixtures of sugars with various drugs to make a suspension. The various sinks illustrated in the Examples section of the '260 patent were shown to vary widely in their capacity to control the various types of drugs tested, demonstrating the unpredictable nature of the selection of release controlling substances, such as sugar or mixtures of sugars, suitable for controlling the release of any given active agent or combination of active agents from microcapsules.

Several of the examples of sinks illustrated in the Examples section of the '260 patent contained sorbitol or mixtures of sorbitol and at least one other release controlling substance. However, the concentration of sorbitol in each such ready-to-use suspension solution was so high that it would be likely to cause diarrhea if administered to a human being. For a discussion of the dangers of this side effect of the oral administration of large quantities of sorbitol, see “Petition for Regulatory Action to Revise the Labeling Requirements for Foods Containing Sorbitol”, Center for Science in the Public Interest (submitted to the U.S. Department of Health and Human Services Food and Drug Administration on Sep. 27, 1999), published on the Internet, at “http://www.cspinet.org/foodsafety/labeling_sorbitol.html”.), incorporated by reference herein.

Numerous oxazolidinone compounds have been reported having therapeutically and/or prophylactically useful antibiotic or antimicrobial, in particular antibacterial, effect. Among such oxazolidinone compounds are those illustratively disclosed in the following patents, each of which is individually incorporated herein by reference: U.S. Pat. No. 5,164,510 (Brickner); U.S. Pat. No. 5,231,188 (Brickner); U.S. Pat. No. 5,565,571 (Barbachyn & Brickner); U.S. Pat. No. 5,627,181 (Riedl et al.); U.S. Pat. No. 5,652,238 (Barbachyn et al.); U.S. Pat. No. 5,688,792 (Barbachyn et al.); U.S. Pat. No. 5,698,574 (Riedl et al.); and U.S. Pat. No. 6,069,145 (Betts).

Compounds disclosed in above-cited Barbachyn et al. '792 include, for example, the compound (S)—N-[[3-[3-fluoro-4-(4-morpholinyl)phenyl]-2-oxo-5-oxazolidinyl]methyl]acetamide, also referred to herein as linezolid. Linezolid has the structure shown in formula (I):

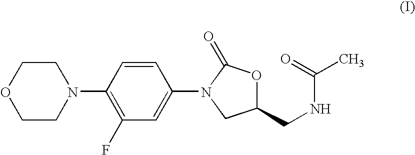

and is in commercial use as a medicament under the trademark ZYVOX® of Pharmacia Corporation.

Linezolid exhibits strong antibacterial activity against gram-positive organisms including those of the following genera: Staphylococcus (e.g., Staphylococcus aureus, Staphylococcus epidermidis), Streptococcus (e.g., Streptococcus viridans, Streptococcus pneumoniae), Enterococcus, Bacillus, Corynebacterium, Chlamydia and Neisseria. Many such gram-positive organisms have developed significant levels of resistance to other antibiotics.

Like other oxazolidinones, linezolid has a very bad taste upon oral administration. A commercial formulation of ZYVOX for oral suspension is an orange-flavored granule/powder for constitution into a suspension with a concentration of 100 mg of linezolid per 5 ml. Sucrose is the only sugar included in the oral suspension formulation. See Physician's Desk Reference, 57^{th} edition, pub. by Thompson, p. 2800 (2003). Unfortunately, even with the orange flavoring, sweeteners, and other inactive ingredients in the formulation, the current oral formulation of linezolid described above has an objectionable taste.

Microencapsulation has been used to mask the taste of linezolid. See, for example, International Publication Number WO 015248 A2 (EURAND AMERICA, INC.), incorporated by reference herein. However, the effectiveness of microencapsulation or of any other known coating method to mask the taste of linezolid or other oxazolidinones is limited. Linezolid tends to leak out of microcapsules or other coated drug particles, into the surrounding suspension medium or into the mouth of a subject after oral administration, leaving an objectionable taste in the mouth of the subject.

What is needed is an oral formulation of linezolid that does not have an objectionable taste. Such a formulation is more likely to be accepted by any subject who can take in liquids orally; thus, making it possible to treat or prevent gram-positive bacteria infections in a greater variety of subjects than would be possible by administration any solid dosage form.

### BRIEF SUMMARY OF THE INVENTION

In one aspect, the present invention relates to dry formulations of coated oxazolidinone particles which, when suspended in an aqueous solution, inhibit mass transport of the oxazolidinone from the particles into the suspension. In this embodiment, the invention specifically relates to a dry formulation comprising at least one dose of coated oxazolidinone particles, each of the particles comprising a core comprising an oxazolidinone and a polymer film at least partially coating the core, a mixture of sugars comprising sorbitol and at least one other sugar, the sorbitol being present in a non-diarrheogenic amount per dose of coated oxazolidinone particles.

In another embodiment, the present invention relates to a suspension of the dry formulation in an aqueous solution.

In another embodiment, the present invention is a method of treating or preventing a gram-positive bacterial infection in a subject comprising orally administering at least one dose of a suspension of the present invention to the subject.

BRIEF DESCRIPION OF THE DRAWING(S)

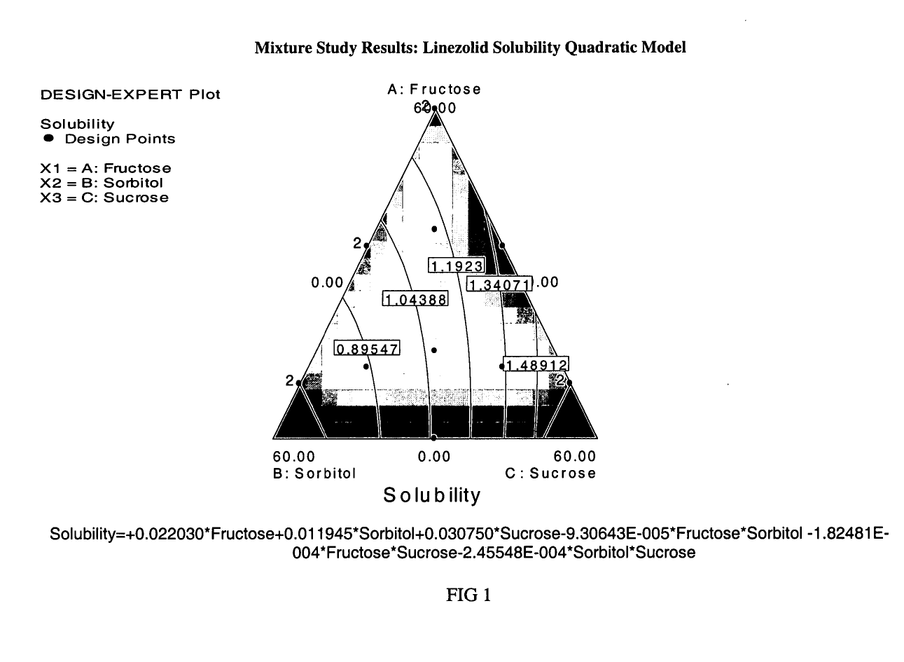

FIG. 1 is a graph produced using a quadratic model, showing linezolid solubility results obtained during a D-optimal statistically designed mixture study with three sugars (fructose, sorbitol, and sucrose), as described in Example 3.

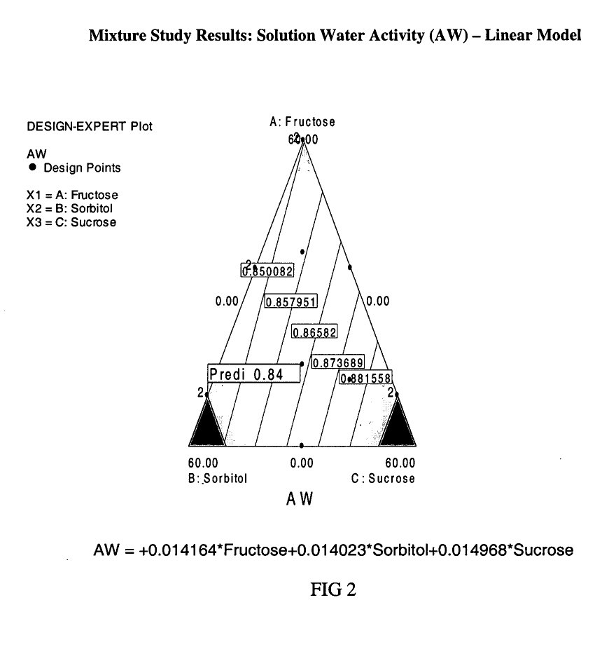

FIG. 2 is a graph produced using a linear model, showing solution water activity (“AW”) results obtained during a D-optimal statistically designed mixture study with three sugars (fructose, sorbitol, and sucrose), as described in Example 3.

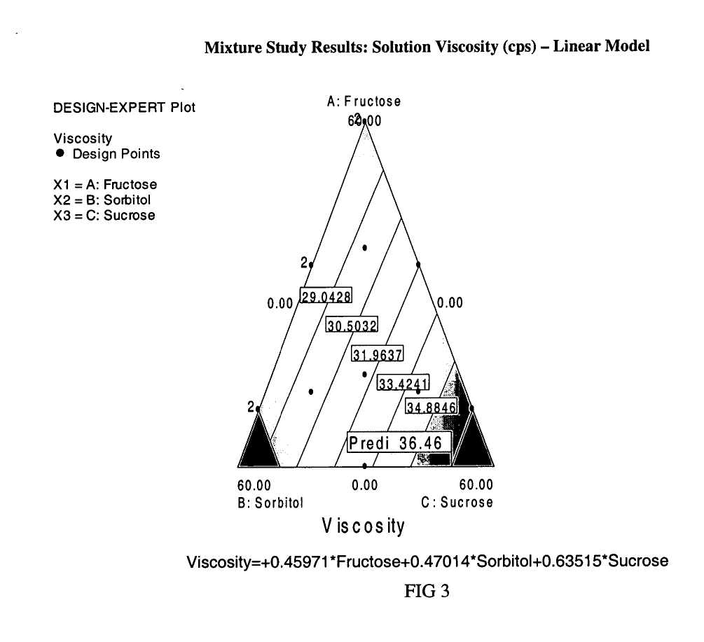

FIG. 3 is a graph produced using a linear model, showing solution viscosity, in cps, measurements obtained during a D-optimal statistically designed mixture study with three sugars (fructose, sorbitol, and sucrose), as described in Example 3.

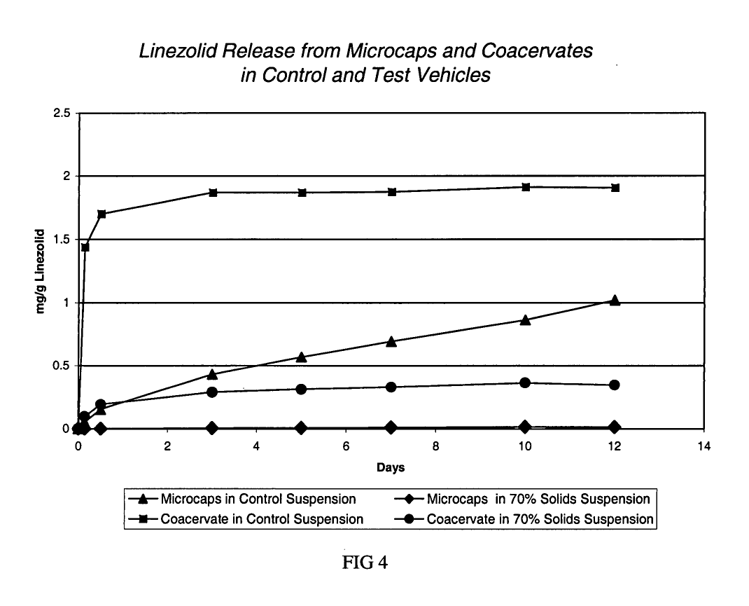

FIG. 4 is a graph showing linezolid release profiles form microencapsulated linezolid particles (“Microcaps”) and coacervated linezolid particles (“Coacervates”) suspended in control and test vehicles, as described in Example 5.

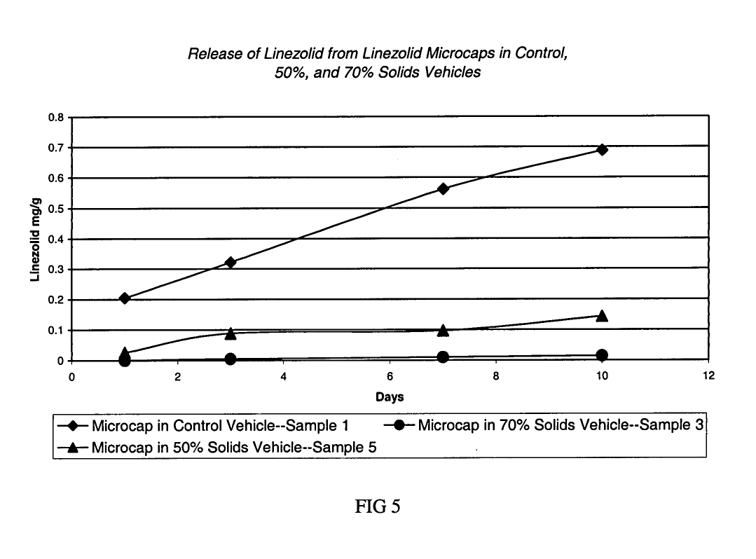

FIG. 5 is a graph showing linezolid release profiles from Microcaps suspended in a control vehicle, or in vehicles with either 50% solids or 70% solids content, as described in Example 8.

### DETAILED DESCRIPTION OF THE INVENTION

The term “coated drug particles”, as used herein, indicates a micron sized core comprising at least one drug in the form of particles, powders, crystals, granules, pellets, and liquid drops, coated at least in part with a polymeric film.

The term “coated linezolid particles” refers to coated drug particles, wherein at least one drug in the core is linezolid.

The term “microencapsulation”, as used herein, refers to a process consisting of coating a micron sized core comprising a drug or combination of drugs with a continuous polymeric film.

The term “microencapsulated linezolid particles”, as used herein, refers to coated linezolid particles, wherein a micron sized core has been coated with a continuous polymeric film. The term “Microcap”, as used herein, refers to a particular type of microencapsulated particle described and defined in the Examples section, below.

The term “coacervation”, as used herein, refers to a process of solubilizing core material comprising a drug and a polymer and allowing the polymer to coat the drug particles through precipitation, with most of the drug being contained within the particles. Examples of coacervation are provided in Reo & Fredrickson, “Tastemasking Science and Technology Applied to Compacted Oral Solid Dosage Forms—Part 2”, Amer Pharm Rev (Fall 2002), pp. 2-13, and in WO 99/52510 (EURAND INTERNATIONAL SPA), both of which are incorporated by reference herein.

The term “coacervated linezolid particles”, as used herein, refers to coated linezolid particles in the form of micron sized coacervates comprising linezolid and a polymer. The linezolid in such particles may not all be coated with the polymer. The term “Coacervate” is used herein to refer to a particular type of coacervated linezolid particle described in the Examples section, below.

The term “oral administration” herein includes any form of delivery of a therapeutic agent or a composition thereof to a subject wherein the agent or composition is swallowed by a subject, regardless of whether the composition is placed in the mouth prior to swallowing. The term “oral administration” includes esophageal administration. Absorption of the agent can occur in any part or parts of the gastrointestinal tract including the mouth, esophagus, stomach, duodenum, ileum and colon.

The term “orally deliverable” herein means suitable for oral administration.

A “subject” herein to which a therapeutic agent or composition thereof can be administered includes a human patient of either sex and of any age, and also includes any nonhuman animal, particularly a domestic or companion animal, illustratively a cat, dog or horse.

The term “dose” herein means an amount of a drug or pharmaceutical formulation to be taken or applied all at one time or in fractional amounts within a given period. In the case of an oral suspension, a dose is an amount of the suspension to be taken orally at once, or in fractions one after another at a given time period.

The term “multidose” as used herein, refers to at least two doses of a drug or pharmaceutical formulation.

The term “multidose sachet” is a container which contains at least two doses of a drug and excipients in a dry formulation.

The term “non-diarrheogenic amount” as used herein, refers to an amount of a substance which, when administered to a subject, does not give rise to diarrhea.

The term “excipient” herein means any substance, not itself a therapeutic agent, used as a carrier or vehicle for delivery of a therapeutic agent to a subject or added to a pharmaceutical composition to improve its handling, storage, disintegration, dispersion, dissolution, release or organoleptic properties or to permit or facilitate formation of a dose unit of the composition into a discrete article such as a capsule or tablet suitable for oral administration. Excipients can include, by way of illustration and not limitation, diluents, disintegrants, binding agents, adhesives, wetting agents, polymers, lubricants, glidants, substances added to mask or counteract a disagreeable taste or odor, flavors, dyes, fragrances, and substances added to improve appearance of the composition.

As used herein, the term “stable suspension” refers to a suspension of particles wherein the particles remain in suspension, with no visible floating or sedimentation, for at least 24 hours with no mixing after an initial suspension step.

The term “substantially homogeneous suspension”, as used herein, refers to a suspension of solid material in a solution, such as a suspension of microencapsulated drug in a solution, wherein substantially uniform dosing is possible throughout the suspension.

The term “viscosity enhancing substance”, as used herein, refers to substances which dissolve in water and which increase in density and viscosity, allowing solid particles to be suspended therein.

The oxazolidinone component of the coated oxazolidinone particles of the dry formulation of the present invention comprises an oxazolidinone moiety as part of its chemical structure. Preferred oxazolidinones are compounds having formula (II):

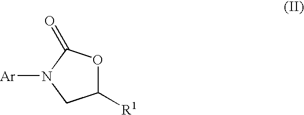

where Ar is an optionally substituted aryl or heteroaryl group and R1 is a group selected such that the compound of formula (II) falls within the scope of compounds generically or specifically disclosed in any of the following patents, each of which is individually incorporated herein by reference: U.S. Pat. No. 5,164,510 (Brickner); U.S. Pat. No. 5,231,188 (Brickner); U.S. Pat. No. 5,565,571 (Barbachyn & Brickner); U.S. Pat. No. 5,627,181 (Riedl et al.); U.S. Pat. No. 5,652,238 (Barbachyn et al.); U.S. Pat. No. 5,688,792 (Barbachyn et al.); U.S. Pat. No. 5,698,574 (Riedl et al.); and U.S. Pat. No. 6,069,145 (Betts).

More preferably, Ar is an optionally substituted 5- or 6-membered aryl or heteroaryl ring having 0 to 3 heteroatoms selected from nitrogen, oxygen and sulfur.

Still more preferably, Ar is a group

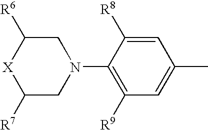

where X is O, S, SO, SO₂, SNR⁴, S(O)NR⁴, NR⁴ or NC(O)CH₂OR⁴, where R⁴ is selected from hydrogen, R⁵ and —C(O)R⁵ groups where R⁵ is C1-8 hydrocarbyl optionally substituted with one or more hydroxy, fluorine or chlorine groups; R⁶ and R⁷ are independently selected from hydrogen, methyl and cyano groups; and R⁸ and R⁹ are independently selected from hydrogen, fluorine and chlorine atoms. Most preferably R⁶ and R⁷ are hydrogen, and one of R⁸ and R⁹ is fluorine and the other of R⁸ and R⁹ is hydrogen.

Preferably R¹ is a group —(CH₂)ₙN(R₂)COR³ where n is 1 to 3, and R² and R³ are independently selected from hydrogen and C1-8 hydrocarbyl optionally substituted with one or more hydroxy, fluorine or chlorine groups.

Examples of preferred oxazolidinones are compounds selected from linezolid, N-((5S)-3-(3-fluoro-4-(4-(2-fluoroethyl)-3-oxopiperazin-1-yl)phenyl)-2-oxooxazolidin-5-ylmethyl)acetamide, (S)—N-[[3-[5-(3-pyridyl)thiophen-2-yl]-2-oxo-5-oxazolidinyl]methyl]acetamide, (S)—N-[[3-[5-(4-pyridyl)pyrid-2-yl]-2-oxo-5-oxazolidinyl]methyl]acetamide hydrochloride and N-[[(5S)-3-[4-(1,1-dioxido-4-thiomorpholinyl)-3,5-difluorophenyl]-2-oxo-5-oxazolidinyl]methyl]acetamide. In one especially preferred embodiment, the oxazolidinone is linezolid.

Oxazolidinone compounds used in compositions of the invention can be prepared by any one of a number of known processes. In the case of linezolid, suitable processes include those described in the following patents, each of which is individually incorporated herein by reference: above-cited U.S. Patent No. 5,688,792 (Barbachyn et al.); U.S. Pat. No. 5,837,870 (Barbachyn et al.); and International Patent Publication No. WO 99/24393 (PHARMACIA & UPJOHN COMPANY).

The present invention is illustrated herein with particular reference to linezolid; however, it will be understood that any other oxazolidinone antimicrobial drug can, if desired, be substituted in whole or in part for linezolid, with appropriate adjustment in concentration and dosage ranges, in the compositions and methods herein described.

The composition of sugars in a suspension vehicle can dramatically affect mass transport of a drug, even from coated particles into a vehicle. The capacity of any given sugar or mixture of sugars to inhibit or promote mass transport of any given drug into a vehicle depends upon a variety of factors, including the chemical and physical characteristics of the drug. The dry formulation of the present invention includes a mixture of sugars comprising sorbitol and at least one other sugar. The at least one other sugar is preferably selected from the group consisting of poly- or oligosaccharides, such as dextrose; disaccharides, such as saccharose, maltose, or lactose; or monosaccharides, such as glucose, fructose, galactose, mannose, or xylitol; or a mixture of two or more of the above. The at least one other sugar is more preferably sucrose or fructose, or a mixture of sucrose or fructose.

The mixture of sugars is preferably about 40% to about 90%, more preferably about 45% to about 75%, even more preferably about 45% to about 55%, most preferably about 50% by weight of the dry formulation.

In the mixture of sugars the weight ratio of sorbitol to the at least one other sugar is preferably at least about 0.5:1, more preferably at least about 1:1, even more preferably at least about 1.5:1. When the at least one other sugar is sucrose, the dry formulation preferably comprises about 20% to about 35% by weight sucrose, and about 25% to about 40% by weight sorbitol. The at least one other sugar is preferably sucrose. When the at least one other sugar is a mixture of sucrose and fructose, the dry formulation preferably comprises about 20% to about 30% by weight sucrose, about 30% to about 40% by weight sorbitol, and about 5% to about 15% by weight fructose.

The selection of sugars and of relative amounts of sorbitol and the at least one other sugar in the sugar mixture depends upon the degree to which inhibition of release of the drug from the coated oxazolidinone particle is desired, upon the characteristics of the drug itself, and upon the relative importance of minimizing dissolution time of the dry formulation in an aqueous solution. When the oxazolidinone is linezolid, and a dissolution time of less than 4 minutes is desired, the dry formulation preferably comprises about 20% sucrose by weight and about 30% sorbitol by weight, for a total of about 50% by weight of the dry formulation being sugars, is particularly preferred.

The total amount of sorbitol per dose of coated oxazolidinone particles in the mixture is a non-diarrheogenic amount. The amount of sorbitol that is non-diarrheogenic for any given subject depends upon a variety of factors, including the age and species of the subject, and whether the subject is diabetic. The amount of sorbitol per dose of the coated oxazolidinone particles in the formulations and suspensions of the present invention is preferably less than about 40 g, more preferably less than about 30 g, even more preferably less than 10 g.

The mixture of sugars in the formulations of the present invention, particularly the preferred mixtures described above, have a surprisingly synergistic effect in inhibiting the mass transport of oxazolidinones, such as linezolid, from the coated oxazolidinone particles in suspensions of the dry formulation of the present invention. (See, for example, Examples 2-4, below).

The mixture of sugars in the dry formulation of the present invention can inhibit the transport of oxazolidinone into an aqueous suspension vehicle, even if the oxazolidinone is in the form of solid, uncoated, particles. However, the oxazolidinone is preferably present in the dry formulation and in the suspensions of the present invention in the form of coated particles. The polymer coating of coated oxazolidinone particles and the mixture of sugars in the formulation inhibit transport of the oxazolidinone into the suspension vehicle to a considerably greater extent than either has the capacity to inhibit, when used in the absence of the other.

The coating of the coated oxazolidinone particles used in the dry formulations, suspensions, and methods of the present invention preferably reduces the availability of the oxazolidinone compared to a suspension of uncoated oxazolidinone, while not adversely impacting the bioavailability of the oxazolidinone. The polymer coating preferably coats at least 70% of the oxazolidinone in the core of each coated oxazolidinone particle, more preferably at least 80% of the oxazolidinone in the core, even more preferably at least 90% of the oxazolidinone in the core. In one preferred embodiment of the present invention, the coated oxazolidinone particles are coacervated oxazolidinone particles. In another preferred embodiment, the coated oxazolidinone particles are microencapsulated oxazolidinone particles.

The coated oxazolidinone particles of the present invention can suitably be produced by any one of a number of known means of coating of core particles, including means described in Reo & Fredrickson, “Tastemasking Science and Technology Applied to Compacted Oral Solid Dosage Forms—Part 2, Amer Pharm Rev (Fall 2002), pp. 2-13, incorporated by reference herein. Suitable means of microencapsulation for use in producing the suspensions and in practicing the methods of the present invention are disclosed in the above-cited article by Reo & Fredrickson, and in U.S. Pat. No. 3,196,827 (Wurster et al.), U.S. Pat. No. 3,253,944 (Wurster et al.), U.S. Pat. No. 3,415,758 (Powell et al.), U.S. Pat. No. 3,155,590 (Miller et al.), U.S. Pat. No. 3,341,416 (Anderson et al.), U.S. Pat. No. 5,008,117 (Calanchi et al.), U.S. Pat. No. 6,261,602 B1 (Calanchi et al.), and U.S. Pat. No. 6,139,865 (Friend et al.), all of which are incorporated herein by reference. The particular coating method selected depends upon the physical characteristics of the oxazolidinone to be microencapsulated. For example, when the oxazolidinone is in the form of a liquid, the polymer film and method used to coat the oxazolidinone in the film is preferably one that is effective in containing the liquid in both a dry formulation and in a suspension medium. In contrast, when the oxazolidinone is in the form of particles or crystals, it can be coated with any one of a wide variety of different pharmaceutically acceptable polymer films. The oxazolidinone in the formulations of the present invention is preferably in the form of oxazolidinone particles or oxazolidinone crystals, more preferably in the form of oxazolidinone particles.

Hydrophobic polymers suitable for use as the polymer film of the coated particles used in the present invention include, but are not limited to, vinyl acetate, vinyl chloride, vinyl carbonate, methacrylic acid, polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate. Ethyl cellulose, cellulose acetate phthalate methacrylic acid, and polymethacrylic acid copolymer are preferred, with methacrylic acid, and polymethacrylic acid copolymers being particularly preferred.

Some hydrophobic polymers, such as ethylcellulose can be processed in such a way that they form a microparticulate coacervate with oxazolidinones, such as linezolid, another form of coated drug particles suitable for use in the formulations and suspensions of the present invention. Some such coacervates will completely encapsulate the drug. However, to ensure complete encapsulation, it is possible to add a coating of a second polymer to the coacervate.

The pharmaceutically acceptable polymer film suitably comprises at least two layers, such as an inner layer with the capacity to delay drug release, such as ethylcellulose or a coacervate of an oxazolidinone and ethylcellulose, and an outer hydrophobic polymer layer, such as polymethacrylate, that dissolves on a pH dependent basis. The method used to produce the microencapsulated oxazolidinone particles, included in one embodiment of the dry formulations or suspensions of the present invention, depends upon the physical characteristics of the oxazolidinone and of the polymer used to produce the polymer film. For suitable methods for use in producing the microencapsulated drug particles included in the formulations and suspensions of the present invention, see Reo & Fredrickson, supra, and WO 99/52510 (EURAND INTERNATIONAL SPA), all of which are incorporated by reference herein. Reo and Fredrickson (supra), specifically, review and evaluate numerous polymer film and substrate particle, crystalline, and matrix configurations described in the literature. Any one of the configurations utilizing hydrophobic polymer films disclosed therein would be suitable for use in the methods and suspensions of the present invention.

Regardless of whether the coated particles include one or more coating layers of hydrophobic polymer, at least one layer of polymer film coating preferably includes a plasticizer deposited thereon or incorporated therein. When the coated oxazolidinone particles include at least two coating layers of polymer film, the outer layer is preferably plasticized pharmaceutical grade shellac, Colorcon Opadry, or a plasticized hydroxypropylmethylcellulose formulation.

The hydrophobic polymer coating of a coated drug particle, particularly when the coated drug particle is microencapsulated, can delay release of the drug in suspension until after administration to a subject. When administration is oral and the drug is one with an offensive taste, microencapsulation can mask the offensive taste by delaying release until after the drug formulation has passed through the mouth of a subject. Even partial coating of a drug with a hydrophobic polymer coating, as described above, can delay release of the drug, both in suspension and after administration to a subject, decreasing any offensive drug taste. Such factors are particularly important when the subject is one likely to reject offensive tasting drugs. At least one drug in the core of the coated drug particles used in the formulations, suspensions, and methods of the present invention is linezolid, an oxazolidinone with a particularly offensive taste when taken orally.

The coated oxazolidinone particles in the dry formulations and suspensions of the present invention, taken together, preferably contain at least one dose, a therapeutic amount of the drug. How much of the oxazolidinone constitutes a therapeutic amount for a given subject is dependent inter alia on the type of oxazolidinone and on the body weight of the subject. When the subject is a child or a small animal (e.g., a dog), and the oxazolidinone is linezolid, an amount of relatively low in the preferred range of about 5 mg/kg to about 10 mg/kg dosed 3 times daily, for example would be suitable for administration. When the subject is an adult human or a large animal (e.g., a horse), achievement of equivalent blood serum concentrations of linezolid are likely to require dose units containing a relatively greater amount of the drug. For an adult human, a therapeutically effective amount of linezolid in a composition of the present invention is typically about 400 to about 600 mg dosed twice daily. Biologically equivalent doses of other oxazolidinones can suitably be administered.

The amount of drug in a given dosage form can be selected to accommodate the desired frequency of administration used to achieve a specified daily dosage. The amount of the unit dosage form of the composition that is administered and the dosage regimen for treating the condition or disorder will depend on a variety of factors, including the age, weight, sex and medical condition of the subject, the severity of the condition or disorder, the route and frequency of administration, and the particular drug selected, and thus may vary widely. One or more dosage forms can be administered up to about 6 times a day. One or more dosage forms of the present invention are more preferably suitable for administration up to about 3 times per day.

The core of each coated oxazolidinone particle preferably consists solely of an oxazolidinone, such as linezolid, or of a mixture of the oxazolidinone and at least one other drug. Specifically, it is preferable to minimize the number of excipients in the core, in order to minimize any possible interference with taste masking of the oxazolidinone. However, it is suitable to include one or more excipients the core of the coated oxazolidinone particles, such as provided below.

In one embodiment of the present invention, the core of the coated oxazolidinone particles further comprises at least one core excipient selected from the group consisting of pharmaceutically acceptable diluent, binding agent, adhesive, wetting agent, lubricant, plasticizer, and anti-adherent agent. Through selection and combination of core excipients, compositions can be provided exhibiting improved performance with respect to, among other properties, efficacy, bioavailability, clearance time, stability, compatibility of drug and excipients, safety, dissolution profile, and/or other pharmacokinetic, chemical and/or physical properties. Preferably, the amount and number of excipients in the core is minimized in order to avoid adversely affecting the taste or mouth feel of the suspension, upon oral administration.

When at least one core excipient is a diluent, the diluent is suitably lactose, including anhydrous lactose and lactose monohydrate; a starch, including directly compressible starch and hydrolyzed starches (e.g., Celutab™ and Emdex™); mannitol; sorbitol; xylitol; dextrose (e.g., Cerelose™ 2000) and dextrose monohydrate; dibasic calcium phosphate dihydrate; sucrose-based diluents; confectioner's sugar; monobasic calcium sulfate monohydrate; calcium sulfate dihydrate; granular calcium lactate trihydrate; dextrates; inositol; hydrolyzed cereal solids; amylose; celluloses including microcrystalline cellulose, and amorphous cellulose (e.g., Rexcel™) and powdered cellulose; calcium carbonate; glycine; bentonite; polyvinylpyrrolidone; and combinations of any of the above.

Microcrystalline cellulose is a preferred diluent. This diluent is chemically compatible with the oxazolidinone. Inclusion of microcrystalline cellulose in the core of coated drug particles can improve hardness and/or disintegration time of the particles. Microcrystalline cellulose typically provides compositions having suitable release rates of drugs admixed therewith, stability, flowability, and/or drying properties at a relatively low diluent cost.

The core of coated drug particles optionally comprise at least one pharmaceutically acceptable binding agent or adhesive as a core excipient. Such binding agents and adhesives preferably impart sufficient cohesion to the core while allowing the particles to disintegrate and the oxazolidinone or mixture of the oxazolidinone and at least one other drug to be absorbed after the coated drug particles pass through the mouth and into the remainder of the gastrointestinal tract of a subject, after ingestion. Suitable binding agents and adhesives include, either individually or in combination, acacia; tragacanth; sucrose; gelatin; glucose; starches such as, but not limited to, pregelatinized starches (e.g., National™ 1511 and National™ 1500); celluloses such as, but not limited to, microcrystalline cellulose, methylcellulose and carmellose sodium (e.g., Tylose™); alginic acid and salts of alginic acid; magnesium aluminum silicate; PEG; guar gum; polysaccharide acids; bentonites; povidone, for example povidone K-15, K-30 and K-29/32; polymethacrylates; HPMC; hydroxypropylcellulose (e.g., Klucel™); and ethylcellulose (e.g., Ethocel™).

The coated oxazolidinone particles optionally comprise one or more pharmaceutically acceptable disintegrants as excipients. Suitable disintegrants include, either individually or in combination, starches, including sodium starch glycolate (e.g., Explotab™ of PenWest) and pregelatinized corn starches (e.g., National™ 1551, National™ 1550, and Colorcon™ 1500), clays (e.g., Veegum™ HV), celluloses such as purified cellulose, microcrystalline cellulose, methylcellulose, carboxymethylcellulose and sodium carboxymethylcellulose, croscarmellose sodium (e.g., Ac-Di-Sol™ of FMC), alginates, crospovidone, and gums such as agar, guar, locust bean, karaya, pectin and tragacanth gums.

The coated oxazolidinone particles optionally comprise at least one pharmaceutically acceptable wetting agent as a core excipient. Non-limiting examples of plasticizers suitable for use as wetting agents in compositions of the invention include quaternary ammonium compounds, for example benzalkonium chloride, benzethonium chloride and cetylpyridinium chloride, dioctyl sodium sulfosuccinate, polyoxyethylene alkylphenyl ethers, for example nonoxynol 9, nonoxynol 10, and octoxynol 9, poloxamers (polyoxyethylene and polyoxypropylene block copolymers), polyoxyethylene fatty acid glycerides and oils, for example polyoxyethylene (8) caprylic/capric mono- and diglycerides (e.g., Labrasol™ of Gattefossé), polyoxyethylene (35) castor oil and polyoxyethylene (40) hydrogenated castor oil; polyoxyethylene alkyl ethers, for example polyoxyethylene (20) cetostearyl ether, polyoxyethylene fatty acid esters, for example polyoxyethylene (40) stearate, polyoxyethylene sorbitan esters, for example polysorbate 20 and polysorbate 80 (e.g., Tween™ 80 of ICI), propylene glycol fatty acid esters, for example propylene glycol laurate (e.g., Lauroglycol™ of Gattefossé), sodium lauryl sulfate, fatty acids and salts thereof, for example oleic acid, sodium oleate and triethanolamine oleate, glyceryl fatty acid esters, for example glyceryl monostearate, sorbitan esters, for example sorbitan monolaurate, sorbitan monooleate, sorbitan monopalmitate and sorbitan monostearate, tyloxapol, and mixtures thereof.

The core of the coated particles optionally comprises at least one pharmaceutically acceptable lubricant, as a core excipient. Suitable lubricants include, either individually or in combination, glyceryl behapate (e.g., Compritol™ 888); stearic acid and salts thereof, including magnesium, calcium and sodium stearates; hydrogenated vegetable oils (e.g., Sterotex™); colloidal silica; talc; waxes; boric acid; sodium benzoate; sodium acetate; sodium fumarate; DL-leucine; PEG (e.g., Carbowax™ 4000 and Carbowax™ 6000); sodium oleate; sodium lauryl sulfate; and magnesium lauryl sulfate. The lubricant is preferably an anti-adherent. Suitable anti-adherents include talc, cornstarch, DL-leucine, sodium lauryl sulfate, colloidal silica, and metallic stearates. Talc is a preferred anti-adherent or glidant used, for example, to reduce formulation sticking to equipment surfaces and also to reduce static in the blend.

The coated oxazolidinone particles included in the dry formulations and suspensions of the present invention are suitably of any size in the micron range. However, the particles are preferably sufficiently small to be suspended in the suspension vehicle and sufficiently large to contain a sufficient amount of oxazolidinone so that a dose of the oxazolidinone will be contained within a reasonable volume for oral administration to a subject. The coated oxazolidinone particles preferably have an average particle size of about 50 microns (hereinafter, “μm”) to about 600 μm, more preferably an average particle size of about 75 μm to about 400 μm, more preferably an average particle size of about 100 μm to about 250 μm, even more preferably an average particle size of about 100 μm to about 180 μm.

In addition to the coated oxazolidinone particles and mixture of sugars, described above, the dry formulation of the present invention optionally further comprises a viscosity enhancing substance. When present at an appropriate concentration, the viscosity enhancing substance acts as a suspension enhancer. The viscosity of the suspension produced by the combination of an aqueous solution, such as water, to the dry formulation is preferably sufficiently low that the suspension has good flow characteristics, in order to facilitate oral administration.

The viscosity enhancing substance is preferably selected from the group consisting of an alginate, carageenin, agar-agar, tragacanth gum, xanthan gum, guar gum, caroba gum, karaya gum, modified corn starch, carboxymethyl cellulose, and crystalline cellulose alone or in combination with other hydrocolloids. The viscosity enhancing substance preferably comprises xanthan gum or a mixture of xanthan gum and at least one other viscosity enhancing substance, such as microcrystalline cellulose and carboxymethylcellulose. The viscosity enhancing substance is most preferably a mixture of xanthan gum, microcrystalline cellulose, and carboxymethylcellulose.

The formulations of the present invention preferably further comprise a taste-masking substance other than the mixture of sugars. At least one taste-masking substance is suitably an artificial sweetener, a flavoring agent, or a combination of a sugar and at least one artificial sweetener or flavoring agent.

Any flavoring agent is suitable for inclusion in the formulations of the present invention, when the drug is suitably taste-masked in the absence of the flavoring agent. Flavoring agents are also suitable for use, that mask detectable objectionable tastes or other unpleasant flavors found to be present in some suspensions of dry formulations of the present invention, in the absence of such flavoring agents.

The coated oxazolidinone particles in the dry formulation of the present invention are preferably suspended within about ten (10) minutes, more preferably within about five (5) minutes, more preferably within about four (4) minutes, more preferably within about three (3) minutes, even more preferably within about one (1) minute of being combined with an aqueous solution.

The suspension of the present invention is produced by combining the dry formulation of coated oxazolidinone particles described herein above with an aqueous solution and suspending the particles therein. The aqueous solution is any aqueous solution suitable for oral administration to a subject, such as a buffer or saline solution, more preferably, water.

In another embodiment, the present invention relates to a method of using a suspension of a dry formulation of the present invention, wherein the coated drug particles are coated oxazolidinone antibiotic drug particles, to treat or prevent an a gram-positive infection in a subject. The oxazolidinone antibiotic drug is preferably linezolid. The method comprises orally administering at least two doses of a suspension of a dry formulation of the present invention to a subject who either has a gram-positive infection or who is at risk of contracting a gram-positive infection. Preventative use is appropriate, for example, prior to or after invasive surgery, or after a subject has contracted an open wound that has not yet become infected. Preferred features and optional suitable components of the suspension suitable for use in the method of the present invention are described herein above.

The present invention is further illustrated by the following examples. These examples are intended to be illustrative of the invention and should not be used to limit or restrict its scope.

### EXAMPLES

The following examples illustrate one or more of the embodiments of the invention described above.

### Example 1

### Assay Methods Used

A study was conducted to investigate the effect of non-electrolyte (sugar-based polyols) and electrolyte solutions on linezolid solubility, water activity, viscosity and linezolid mass transport from microencapsulated linezolid. The study was implemented in three phases: screening, optimization, and mass transport. The primary goal of the study was to identify a suspension vehicle for microencapsulated linezolid particles that would reduce the quantity of dissolved linezolid in the suspension compared to known suspension vehicles.

The following specific types of assays were performed in one or more of each of the phases of the study:

1. Water Activity

A Pawkit water activity meter uses a dielectric humidity sensor to measure the water activity (a_{w}) of a sample. The unit is accurate to ±0.02 a_{w}. The water activity meter was calibrated, using a 6.0 molal NaCl solution, before each new set of measurements was taken. Each sample was measured in duplicate.

2. Viscosity Measurements

In the screening and optimization phases, described below, viscosity measurements were made using a Bohlin Rheometer, under the following conditions: a 10.0 pascal sheer stress and a continuous, single sweep type at a temperature of 25.0° C.

3. Linezolid Equilibrium Saturation Solubility Measurements

Bulk linezolid and test solutions were mixed in approximately a 20 mg linezolid/ml solution concentration, which is the linezolid concentration in the commercialized oral suspension formulation. The linezolid equilibrium saturation solubility is about 3 mg/ml. Approximately 200 mg of bulk drug was weighed into 20 ml glass vials. The vials were then filled approximately half-full. The vials were then shaken for at least 24 hours. Using a Iml syringe, an amount of the linezolid-saturated solution slightly greater than 1 ml was withdrawn from the middle of the vial. All bubbles were removed from the syringe by tapping the sides and forcing the bubbles out through the end. Approximately 0.3mls of saturated solution was pushed through the syringe and 0.45 μm filter unit to saturate the filter. The next 0.3 ml aliquot was put into each 10 ml volumetric flask. The volumetric flasks were diluted using a mobile phase solution, and a sample of each diluted solution was analyzed by HPLC. All concentration results were reported in mg/ml.

4. Release Rate Studies

Appropriate amounts of each type of coated linezolid particle tested were added to each vehicle in a vial, to attain a theoretical 20 mg/ml linezolid suspension. The vials were shaken to disperse the particles in the vehicle, and then shaken prior to each sample time point.

For each time point, the vial was shaken vigorously. However, the particles settled to the bottom of the vial prior to sample removal. Using a 5 ml syringe, approximately 4 ml of the mixture was withdrawn. A filter unit was attached to the end of the syringe and 1 ml of the mixture was flushed through the filter to saturate it. Approximately 1-ml of the filtered solution was put into a tared volumetric flask and weighed. The volumetric flask was diluted using an aqueous solution of 0.1% trifluoroacetic acid. HPLC parameters used in this study were the same as described in the saturation solubility measurements, above. The linezolid found in solution during this study was used to determine the amount linezolid (mg) present in 1 g of solution and was reported in units of mg/g.

### Example 2

### Screening Phase

During the screening phase, various concentrations of aqueous solutions of each of several different sugars and electrolytes were evaluated. Selection of the suspension excipients used in the study was based on structural, ionic strength, solubility difference and practical considerations. Criteria used included molecular weight, charge density, hydroxyl functional group density and pharmaceutical acceptance. Sugars and electrolytes evaluated included sucrose (disaccharide composed of 6-carbon and 5-carbon monosaccharides), glucose (6-carbon monosaccharide and component of sucrose), fructose (5-carbon monosaccharide, keto sugar and component of sucrose), sorbitol (6-carbon sugar alcohol), maltose (disaccharide composed of two 6-carbon monosaccharides), sodium chloride and calcium lactate solutions. The concentration of each solute to be tested was selected based upon its saturation solubility in water. The assay results are summarized in Table 1, below.

TABLE 1

| | | | | Linezolid | | Water | |
|---|---|---|---|---|---|---|---|
| Component | Sample | | | Solubility | | Activity | Viscosity |
| Tested | Number | Molality | % w/w | (mg/ml) | RSD % | (Aw) | (cps) |
| Sucrose | 1 | 5.32 | 65.0 | 1.54 | 2.53 | 0.86 | 119.1 |
| | 2 | 4.68 | 62.0 | 1.74 | 5.84 | 0.88 | 69.6 |
| | 3 | 4.10 | 58.0 | 1.96 | 5.59 | 0.90 | 44.6 |
| | 4 | 3.61 | 55.0 | 2.04 | 2.60 | 0.91 | 33.1 |
| | 5 | 2.65 | 48.0 | 2.07 | 2.22 | 0.94 | 15.2 |
| | 6 | 1.13 | 28.0 | 2.46 | 1.42 | 0.98 | 9.9 |
| | 7 | 0.52 | 15.0 | 2.55 | 1.16 | 0.99 | 8.7 |
| Sorbitol | 8 | 9.88 | 64.0 | 0.54 | 3.40 | 0.81 | 48.7 |
| | 9 | 9.08 | 62.0 | 0.56 | 1.28 | 0.82 | 39.1 |
| | 10 | 7.78 | 59.0 | 0.65 | 3.06 | 0.85 | 27.3 |
| | 11 | 5.48 | 50.0 | 0.91 | 4.55 | 0.91 | 13.9 |
| | 12 | 3.80 | 41.0 | 1.13 | 3.05 | 0.94 | 7.6 |
| | 13 | 1.87 | 25.0 | 1.68 | 3.29 | 0.98 | 9.4 |
| | 14 | 0.93 | 15.0 | 2.12 | 3.30 | 0.99 | 8.4 |
| Fructose | 15 | 16.57 | 75.0 | 0.70 | 7.56 | 0.70 | 429.1 |
| | 16 | 14.75 | 73.0 | 0.78 | 2.25 | 0.74 | 243.2 |
| | 17 | 12.91 | 70.0 | 0.86 | 1.40 | 0.76 | 134.7 |
| | 18 | 9.33 | 63.0 | 0.99 | 0.85 | 0.88 | 52.5 |
| | 19 | 6.16 | 52.0 | 1.34 | 2.14 | 0.93 | 16.5 |
| | 20 | 3.20 | 34.0 | 1.84 | 0.88 | 0.99 | 11.3 |
| | 21 | 1.84 | 24.0 | 2.15 | 1.49 | 0.99 | 9.4 |
| Maltose | 22 | 2.16 | 43.0 | 2.45 | 3.30 | 0.96 | 13.6 |
| | 23 | 1.43 | 33.0 | 2.51 | 1.38 | 0.99 | 11.3 |
| | 24 | 0.60 | 17.0 | 2.61 | 1.69 | 1.00 | 8.6 |
| | 25 | 1.97 | 40.0 | 2.69 | 3.37 | 0.97 | 13.0 |
| Dextrose | 26 | 3.62 | 39.0 | 1.61 | 5.10 | 0.95 | 12.8 |
| | 27 | 2.14 | 28.0 | 1.89 | 1.55 | 0.97 | 9.8 |
| | 28 | 1.53 | 22.0 | 2.06 | 1.33 | 0.99 | 9.3 |
| | 29 | 0.68 | 11.0 | 2.29 | 1.97 | 1.00 | 8.1 |
| NaCl | 30 | 0.18 | 1.0 | 2.74 | 2.99 | 0.99 | 7.5 |
| | 31 | 0.11 | 0.7 | 2.80 | 1.91 | 0.99 | 7.4 |
| | 32 | 0.09 | 0.5 | 2.74 | 3.32 | 1.00 | 7.3 |
| | 33 | 0.03 | 0.3 | 2.91 | 3.29 | 1.00 | 7.6 |
| Ca Lactate | 34 | 0.23 | 7.0 | 2.20 | 2.86 | 0.99 | 8.1 |
| | 35 | 0.17 | 5.0 | 2.25 | 1.30 | 1.00 | 7.5 |
| | 36 | 0.10 | 3.0 | 2.41 | 3.21 | 1.00 | 7.4 |
| | 37 | 0.06 | 2.0 | 2.81 | 2.56 | 0.99 | 7.3 |
| Water | 38 | 0.00 | 0.0 | 3.17 | 1.32 | 1.00 | 7.2 |
| | (Control) | | | | | | |

All solutes studied, as described above, were found to lower the linezolid saturation solubility from the measured solubility of 3.17 mg/ml in water. The data summarized in Table 1, above, showed that linezolid was least soluble in sorbitol, fructose, and sucrose solutions (0.54 mg/ml @ 9.9 molal, 0.70 mg/ml @ 16.6 molal and 1.54 mg/ml @ 5.3 molal, respectively). The water activity and viscosity of these solutions were 0.81 and 48 cps, 0.70 and 429 cps, and 0.86 and 119 cps, respectively.

The data in Table 1 illustrates that water activity is not the only predictor of linezolid water solubility. The sorbitol solutions showed the greatest reduction in linezolid water solubility, even though fructose had the lowest water activity. Fructose had the highest water solubility of the group of sugar compounds or electrolytes tested in this Example.

The solution with the highest concentration of fructose studied, 2.16 molal maltose, had the highest solution viscosity of 439 cps. Fructose also had the highest water solubility of the group of sugar-compounds studied (1 gram in 0.3 ml water).

The highest concentration of maltose studied was 2.16 molal. Higher concentrations were not considered because sucrose and sorbitol reduced the linezolid saturation solubility at least as well as maltose at lower concentrations. The saturation solubilities for linezolid at 2.16 molal maltose, 1.13 molal sucrose and 1.87 molal sorbitol were 2.45, 2.45 and 1.68 mg/ml, respectively. The capacity of maltose to reduce the linezolid saturation solubility was less than the capacity of either sorbitol or sucrose.

Dextrose had the lowest saturation solubility in water. The highest concentration studied was 3.62 molal (39% w/w).

In summary, of the excipients tested in this Example, the sugars exerted the highest reduction in linezolid saturation solubility. Furthermore, it was found that linezolid solubility was reduced with increases in sugar saturation solubility. The one exception was sorbitol, a sugar with a relatively lower saturation solubility, which had the greatest linezolid solubility reduction effect.

The water activity results summarized in Table 1, above, followed an expected trend. The water activity decreased as the solute concentrations increased. As the water activity of the solution decreased, the saturation solubility of linezolid decreased. This was especially evident in the sugar solutions that had the highest amount of sugar present—fructose, sucrose, and sorbitol. Electrolyte solutions were too dilute to exert a significant effect on water activity and therefore, linezolid saturation solubility. Another advantage that sorbitol exhibited was the ability to reduce the water activity as compared to fructose, as shown in Table 1.

The viscosity of the solutions was measured to provide an indication/prediction of the relative effect of viscosity on linezolid release rate from microencapsulated linezolid.

### Example 3

### Optimization Phase

D-optimal statistically designed mixture experiment was done with sorbitol, fructose and sucrose, since these agents showed the greatest reduction in water activity and linesolid solubility. The experiment described below was designed to enable the determination of whether the relationship between the components identified in the screening study, above, would be additive or synergistic. It also provided a model for predicting water activity, viscosity, and linezolid solubility for mixtures of the four components, within certain constraints.

A D-optimal mixture design was used to measure the effects of fructose (0-60%), sorbitol (0-50%) and sucrose (0-50%) in water on the solubility, water activity and of linezolid. The combined total of fructose, sorbitol, and sucrose was kept constant 60% w/w total solids. The composition of each sugar in each mixture tested, and results of solubility, water activity, and viscosity tests performed thereon are summarized in Table 2, below:

TABLE 2
Mixture Optimization Study Results (I)

| | | | | Average | Water | |
|---|---|---|---|---|---|---|
| Solution | Fructose | Sorbitol | Sucrose | Solubility | Activity | Viscosity |
| Number | % w/w | % w/w | % w/w | (mg/ml) | (Aw) | (cps) |
| 1 | 13 | 36 | 11 | 0.907 | 0.85 | 31.9 |
| 2 | 0 | 30 | 30 | 1.05 | 0.87 | 30.4 |
| 3 | 60 | 0 | 0 | 1.36 | 0.85 | 28.8 |
| 4 | 16 | 22 | 22 | 1.09 | 0.86 | 28.7 |
| 5 | 13 | 11 | 36 | 1.32 | 0.88 | 33.4 |
| 6 | 35 | 0 | 25 | 1.36 | 0.87 | 36.6 |
| 7 | 38 | 11 | 11 | 1.18 | 0.86 | 27.7 |
| 8 | 10 | 0 | 50 | 1.66 | 0.89 | 49.9 |
| 9 | 10 | 50 | 0 | 0.779 | 0.85 | 29.2 |
| 10 | 35 | 25 | 0 | 0.964 | 0.85 | 29.3 |
| 11 | 60 | 0 | 0 | 1.28 | 0.85 | 25.0 |
| 12 | 10 | 50 | 0 | 0.759 | 0.84 | 29.2 |
| 13 | 10 | 0 | 50 | 1.68 | 0.89 | 37.4 |
| 14 | 35 | 25 | 0 | 0.993 | 0.84 | 26.1 |

The linezolid solubility results from Table 2, above were plotted out using a quadratic model, as shown in FIG. 1. The resulting quadratic predictor best fit to the solubility data showed that sorbitol had a greater effect on solubility reduction than the other two sugars tested. Fructose had the next greatest effect on solubility reduction, followed by sucrose.

Linear predictor equations were found to best fit the water activity and viscosity data. FIG. 2 is the linear plot of the water activity data. FIG. 3 is a linear plot of the viscosity data. FIGS. 2 and 3 show the water activity and viscosity are both additive, and not synergistic. Using the models illustrated in FIGS. 2 and 3, the water activity and viscosity of the mixtures are predicted as weighted sums of the individual components. The water activity and viscosity of any mixture within the experimental space is in the range 0.85 to 0.88 and 29.0 to 36 cps, respectively.

In summary, the data from the mixture experiment demonstrated that solubility reduction by the mixture of the three sugars tested is synergistic; while, the effect of the three sugars on water activity and viscosity are additive. In addition, the data showed that viscosity was too low to affect solubility. Therefore, the D-optimal mixture study results indicated that the mechanism for reduction in the solubility of linezolid in the mixtures tested depends on factors other than water activity alone, factors that are not related to viscosity.

### Example 4

### Optimization Phase, Continued

Based on the mixture experiment results and considering current laxative dose for sorbitol, the linezolid mass transport of microencapsulated linezolid in a mixture of sorbitol:fructose:sucrose (65% w/w total solids) was compared to a control mixture of sucrose:mannitol. Both mixtures contained preservative, electrolyte and buffer. The drug release was measured over 12 days. The drug release data showed that linezolid mass transport rate is greatly reduced (close to zero) for the sorbitol:fructose:sucrose vehicle, which has lower water activity and linezolid solubility. The conclusion of the study is the microencapsulated linezolid mass transport rate in an aqueous medium was reduced by reducing mass transport driving forces. The reduction in mass transport rate is predicted to result in a better tasting product. This study and results obtained therefrom are described in greater detail, below.

Thirteen mixtures were prepared for use in mixture optimization studies, as described in Table 3, below. The composition of the mixtures tested was based upon the results of the mixture experiments performed as described in Example 3, above. Linezolid saturation solubility and water activity of each mixture were measured. Results obtained in each of these tests are also set forth in Table 3. The solubility results are expressed in Table 3 in terms of mean solubility, in mg/ml, and in terms of the standard deviation (“SD”) and relative standard deviation (“RSD”) of each set of solubility measurements.

TABLE 3
Mixture Optimization Study Results (II)

| | | w/w % | w/w % | Constitution | Solubility | | | Water |
|---|---|---|---|---|---|---|---|---|
| Sample | Excipients | Excipient | Solids | Time | Mg/ml | SD | RSD | Activity |
| 1 | CaCl | 3.3 | 3.3 | 10 minutes | 2.81 | 0.07 | 2.3 | 0.98 |
| 2 | Fructose | 30 | 70 | overnight | 0.67 | 0.03 | 5.1 | 0.78 |
| | Sucrose | 10 | | | 0.68 | 0.06 | 8.3 | |
| | Sorbitol | 30 | | | 0.67 | 0.01 | 0.9 | |
| 3 | Sorbitol | 40 | 70 | 2 hours | 0.64 | 0.05 | 7.6 | 0.76 |
| | Fructose | 30 | | | | | | |
| 4 | Sorbitol | 45 | 70 | 2 hours | 0.58 | 0.02 | 3.0 | 0.77 |
| | Fructose | 25 | | | | | | |
| 5 | Sorbitol | 40 | 65 | 2 hours | 0.73 | 0.03 | 4.4 | 0.81 |
| | Fructose | 25 | | | | | | |
| 6 | Mannitol | 13.8 | 13.8 | overnight | 2.36 | 0.03 | 1.4 | 0.95 |
| 7 | Fructose | 34.5 | 44.8 | overnight | 1.62 | 0.04 | 2.5 | 0.99 |
| | Mannitol | 10.3 | | | | | | |
| 8 | Fructose | 28.1 | 37.5 | overnight | 1.84 | 0.04 | 1.9 | 0.96 |
| | Mannitol | 9.3 | | | | | | |
| 9 | Fructose | 25.4 | 36.3 | overnight | 1.85 | 0.00 | 0.1 | 0.96 |
| | Mannitol | 10.8 | | | | | | |
| 10 | Sucrose | 18.5 | 28.4 | 5 minutes | 2.44 | 0.06 | 2.3 | 0.97 |
| | Mannitol | 9.9 | | | | | | |
| 11 | Sucrose | 18.6 | 28.2 | 5 minutes | 2.46 | 0.06 | 2.4 | 0.96 |
| | Mannitol | 8.7 | | | | | | |
| | Na Citrate | 0.34 | | | | | | |
| | NaCl | 0.2 | | | | | | |
| | Citric Acid | 0.2 | | | | | | |
| | Na | 0.27 | | | | | | |
| | Benzoate | | | | | | | |
| 12 | Sucrose | 29.9 | 70.1 | 2 hours | 0.72 | 0.02 | 3.0 | 0.79 |
| | Sorbitol | 29.9 | | | | | | |
| | Fructose | 10 | | | | | | |
| | NaCl | 0.084 | | | | | | |
| | Citric Acid | 0.083 | | | | | | |
| | Na Citrate | 0.14 | | | | | | |
| | Na | 0.11 | | | | | | |
| | Benzoate | | | | | | | |
| 13 | Sucrose | 30 | 70 | overnight | 0.79 | 0.03 | 3.5 | 0.8 |
| | Sorbitol | 30 | | | | | | |
| | Fructose | 10 | | | | | | |

Some of the mixtures in Table 3 included solutions with 70% by weight concentrations, prepared with various combinations of sucrose, sorbitol, and fructose. As illustrated in Table 3, it was found that the 70% concentration mixtures resulted in the greatest reduction in linezolid saturation solubility. Those with the higher concentrations of sorbitol gave even lower solubility results. For example, a solution consisting of 30% sorbitol, 30% fructose, and 10% sucrose gave a linezolid solubility result of 0.672 mg/ml. Another solution consisting of 45% sorbitol and 25% fructose gave a linezolid solubility result of 0.582 mg/ml.

Although high sorbitol solutions exhibited the lowest linezolid saturation solubility results, these solutions were rejected for use in the release rate study. The problems associated with higher concentrations of sorbitol in the body include gastrointestinal effects such as diarrhea, abdominal pain, and bloating. Recent studies suggest that sorbitol has a laxative effect on the body when taken in dosages of as little as 20 grams/day for adults and in dosages greater than 0.5 g/kg body weight in children. For a summary of such studies, see “Petition for Regulatory Action to Revise the Labeling Requirements for Foods Containing Sorbitol”, U.S. Department of Health and Human Services Food and Drug Administration (submitted Sep. 27, 1999), on the Internet, at “http://www.cspinet.org/foodsafety/labeling_sorbitol.html”.) Because of these studies, it was decided to avoid the solutions containing higher concentrations of sorbitol in the remaining Examples, below; avoiding use of concentrations of sorbitol that would exceed the recommended daily allowance suggested in the most recent studies.

A suspension vehicle comprising 30% sorbitol, 30% sucrose, and 10% fructose was selected for use in the mass transport study, described in Example 5, below. the saturation solubility of linezolid in this vehicle was found to be 0.79 mg/ml. The vehicle was selected for potential use in preparation of twice-a-day dosage forms of linezolid for delivery to adults or children.

### Example 5

### Mass Transport Phase

Two different types of microencapsulated linezolid particles, coacervated and coated coacervated particles, were used to study differences in release rates between a control and test suspension vehicle, in the final phase of the study, described below. Both types of particles were produced by Eurand America, Inc. in Vandalia, Ohio. The coacervated linezolid particles comprised linezolid encapsulated by a thermally induced phase separation coacervation process, using ethylcellulose as the encapsulating polymer. The coated coacervated linezolid particles consisted of the coacervated linezolid particles further coated with two more coating layers. The first additional coating layer of was a seal coat of shellac, such as pharmaceutical glaze. The second additional coating layer was Eudragit RL30D and tributyl citrate. The coacervated linezolid particles are hereinafter referred to as “Coacervates.” The coated Coacervates are hereinafter referred to as “Microcaps.” The term Microcaps® is also a brand name of Eurand America, Inc. However, the term is used herein to refer to the specific type of microencapsulated linezolid particles described immediately above, and not to a brand name of any such particles. A detailed description of the methods of production believed to have been used in producing both types of particles used, below, is disclosed in WO 01/52848 (EURAND AMERICA, INC.), incorporated by reference herein.

A new vehicle composition was selected, based on the results of the D-optimal mixture study and study of additional mixtures, in Examples 3 and 4, respectively, above. Practical considerations were also taken into account, including the laxative dose range for sorbitol, sweetness, and cost of goods. Two suspension vehicles were selected for further evaluation, a control vehicle and a test suspension vehicle, using both coated and uncoated coacervate linezolid particles, as described below. The formula for each vehicle is shown in Table 4, below.

TABLE 4

| CONTROL VEHICLE | | | TEST VEHICLE | | |
|---|---|---|---|---|---|
| | | Solids | | | Solids |
| | Solution | % w/w | | Solution | % w/w |
| Ingredient | % w/w | (Dry Basis) | Ingredient | % w/w | (Dry Basis) |
| Sucrose | 18.55 | 65.78 | Sucrose | 29.90 | 42.60 |
| Mannitol | 8.66 | 30.69 | Sorbitol | 29.90 | 42.60 |
| | | | Fructose | 10.00 | 14.20 |
| Sodium citrate | 0.34 | 1.19 | Sodium citrate | 0.14 | 0.20 |
| Citric acid | 0.20 | 0.70 | Citric acid | 0.08 | 0.12 |
| Sodium chloride | 0.20 | 0.69 | Sodium chloride | 0.08 | 0.12 |
| Sodium benzoate | 0.27 | 0.94 | Sodium benzoate | 0.11 | 0.16 |
| Purified water USP | 71.78 | | Purified water USP | 29.80 | |
| Total % Solids in | 28.22 | | Total % Solids in | 70.2 | |
| Solution | | | Solution | | |

The release rate was examined by plotting the linezolid dissolved in the aqueous phase of the dispersion versus the days over which the study was conducted in the two vehicles described in Table 4, above. One lot of Coacervate and one lot of Microcaps, prepared from the same Coacervate lot, were used in the release study.

Approximately 0.212 g of Microcap particles was added to each of 22-5 ml glass vials. About 0.125 g the Microcap particles was added to each of 22-5 ml glass vials. Based on the density of the particles, control vehicle and test vehicle, a mass of each vehicle was added to each vial to make a dispersion equal to linezolid 100 mg/5 ml. Approximately 5.37 g of the control vehicle was added to 11 of the vials containing the Microcap particles and 5.44 g of the control vehicle was added to 11 of the vials containing the Coacervate particles. Approximately 6.41 g of the test vehicle was added to 11 vials containing the Microcap particles and 6.49 g of the test vehicle was added to 11 vials containing the Coacervate particles. Vials were undisturbed after adding the vehicle. Each vial represented a different sample to be taken at a specific time period. The amount of dissolved linezolid was measured at 0.14, 0.5, 3, 5, 7, 10 and 12 days, from different vials.

At each time period, the vial contents were shaken vigorously, and any undissolved solids were allowed to settle to the bottom of the vial before a 4 ml sample of the resulting mixture was withdrawn from the vial to be sampled, using a 5 ml syringe. A 0.45 micron membrane filter unit was attached to the end of the syringe and 1 ml of the mixture was flushed through the filter to saturate it. Next, about 1 ml of the filtered solution was put into a tarred volumetric flask and weighed to avoid bias/error due to air in the sample. A sample of the filtered solution was diluted and analyzed using HPLC to determine the amount of dissolved linezolid in the suspension vehicle. The dissolved linezolid was expressed as mg linezolid per gram of solution.

Results of the study described immediately above are summarized in Table 5, below, a mean linezolid release profile, with results shown in milligrams of linezolid dissolved per gram of solution. FIG. 4 is a plot of the mean linezolid release profile results from Table 5, below.

TABLE 5

| | | Coacervates | | |
|---|---|---|---|---|
| Time | Coacervates & | & | Microcaps & | Microcaps & |
| (Day) | Control Vehicle | Test Vehicle | Control Vehicle | Test Vehicle |
| 0 | 0 | 0 | 0 | 0 |
| 0.27 | 1.435 | 0.095 | 0.051 | 0.000 |
| 1 | 1.700 | 0.189 | 0.152 | 0.000 |
| 3 | 1.868 | 0.288 | 0.431 | 0.005 |
| 5 | 1.867 | 0.312 | 0.569 | 0.007 |
| 7 | 1.873 | 0.325 | 0.693 | 0.010 |
| 10 | 1.910 | 0.362 | 0.864 | 0.014 |
| 12 | 1.905 | 0.345 | 1.021 | 0.013 |

In Control Vehicle:
Linezolid equilibrium saturation solubility = 2.456 mg/ml = 2.428 mg/g solution based on an estimated saturated solution specific gravity = 1.0116 g/ml. Water Activity of the Control Vehicle = 0.96
In Test Vehicle:
Linezolid equilibrium saturation solubility in Test Vehicle 0.719 mg/ml = 0.547 mg/g solution based on an estimated saturated solution specific gravity = 1.3142 g/ml. Water Activity of the Test Vehicle = 0.79

FIG. 4 is a plot of the results from the release summarized in Table 5, above. FIG. 4 and the data in Table 5 show that the release rates for the Coacervate in the control and test suspension vehicles were initially rapid (up to one day) and then approached zero. FIG. 4 shows that the amount of linezolid initially released in the control vehicle was considerably greater than the amount of linezolid initially released in the test vehicle. The equilibrium linezolid released for the Coacervate particles in the control and test vehicle was approximately 79 and 63 percent of the linezolid saturation solubility, respectively. The linezolid release rate from the Microcap particles in the test vehicle was significantly slower than the release rate in the control vehicle (Table 5 and FIG. 4). The release rate was close to zero for the Microcap dispersion in the test vehicle, starting from time zero. After 12 days, the release rate of linezolid from the Microcap into the test solution was very small. Specifically, the slope, or release rate, was 0.0013 mg/ml/day. The release rate, of linezolid from the Microcap particles into the control vehicle was found to be 0.081 mg/ml/day.

The linezolid release rate from the Microcap particles in the test vehicle was significantly slower than in the control vehicle, as shown in Table 5 and illustrated in FIG. 4. The release rate was close to zero for the Microcap particles dispersed in the test vehicle, starting from time zero. In the test vehicle, the Microcap particles had a significant reduction in linezolid mass transport into aqueous phase, a reduction that corresponds to lower water activity observed at the same time points, indicating lower linezolid saturation solubility. Specifically, the water activity for the test and control vehicles were found to be 0.79 and 0.96, respectively. The saturation solubility for the test and control vehicles were found to be 0.547 and 2.428 mg per gram solution, respectively.

The linezolid concentration for the Coacervate particles in the test vehicle remained below the linezolid concentration for the Microcap particles in the control vehicle over the 12 day study period. Use of Coacervate particles suspended in the test vehicle could avoid complex, costly coating steps once thought necessary to taste mask such drugs for oral administration in a suspension format.

### Example 6

### Formulation for Homogeneity & Reduced Dissolution Time

Once a suitable relative proportion of sucrose, sorbitol, and fructose was identified that minimizes mass transport of linezolid from coated particles, such as Coacervate and Microcap particles, as described in Examples 5 , above, the following study was conducted in order to determine a weight percent range of solids in a suspension vehicle formulation that enable formation of a suspension of the particles with substantial homogeneity in a reasonably short period of time.

Several suspension vehicles were prepared by adding varying amounts of Avicel® RC-591 (FMC Corporation) and xanthan gum, as viscosity enhancing substances, prior to the addition of other excipients. Table 6, below, shows the formulation of each oral suspension vehicle prepared at this stage:

TABLE 6

| | Control | Total Solids | Total Solids | Total Solids |
|---|---|---|---|---|
| | Total | 70.2% | 60.9% | 53.7% |
| | Solids | (70% | (60% | (52% |
| | 28.6% | Sugars) | Sugars) | Sugars) |
| Excipients | w/w % | w/w % | w/w % | w/w % |
| Sucrose | 20.01 | 29.9 | 30 | 21 |
| Sorbitol | 0 | 29.9 | 30 | 31 |
| Fructose | 0 | 10 | 0 | 0 |
| Mannitol | 9.5 | 0 | 0 | 0 |
| NaCl | 0.26 | 0.082 | 0.24 | 0.24 |
| Citric Acid | 0.17 | 0.082 | 0.16 | 0.16 |
| Sodium Citrate | 0.29 | 0.14 | 0.27 | 0.27 |
| SodiumBenzoate | 0.19 | 0.11 | 0.18 | 0.18 |
| Xanthan Gum | 0.09 | ** | ** | 0.4 |
| Avicel RC-591 | 0.10 | ** | ** | 0.47 |
| Water* | 68.9 | Varies* | Varies* | 46.1 |
| TOTAL | 100% | 100% | 100% | 100% |

Microcap particles were added to one set of samples of control vehicles prepared as described in Table 6, above, while Coacervate particles were added to another set of the same samples of control vehicles. The amount of each type of particle added was adjusted such that the dosage of linezolid in the final suspension would be 100 mg linezolid per 5 ml suspension,

Table 7, below, summarizes the results of an experiment in which varying amounts of Avicel RC-591 and xanthan gum were added to each of the 53.7% (hereinafter, “50%”), 60.9% (hereinafter, “60%”), and 70.2% (hereinafter, “70%”) solids vehicles prepared as described in Table 6, above. A sample of the 28.6% solids vehicle, described in Table 6, was included as a control.

TABLE 7

| | | Method | w/w % | w/w % | | Constitution | |
|---|---|---|---|---|---|---|---|
| Vehicle | % | of | Xanthan | Avicel | Viscosity | (Dissolution) | |
| Number | Sugars | Prep. | Gum | RC591 | (cps) | Time | |
| 1 | 70% | † | 0.35 | 0.41 | 9100 | >2 | hours |
| 2 | 70% | † | 0.22 | 0.27 | 1760 | >2 | hours |
| 3 | 70% | † | 0.46 | 0.54 | 9100 | >2 | hours |
| 4 | 70% | † | 0.12 | 0.14 | 970 | >2 | hours |
| 5 | 70% | † | 0.57 | 0.68 | 21400 | >2 | hours |
| 6 | 70% | † | 0.17 | 0.2 | 2460 | >2 | hours |
| 7 | 70% | † | 0 | 0 | 170 | >2 | hours |
| 8 | control | ** | 1.5 | 1.8 | 3010 | not | tested |
| 9 | 70% | ‡ | 0.05 | 0.065 | 740 | 45 | min |
| 10 | 70% | ‡ | 0.028 | 0.033 | 510 | 45 | min |
| 11 | 70% | ‡ | 0.013 | 0.016 | 290 | 45 | min |
| 12 | 70% | ‡ | 0.005 | 0.007 | 160 | 45 | min |
| 13 | 70% | ‡ | 0.07 | 0.08 | 1080 | 45 | min |
| 14 | 70% | ‡ | 0.08 | 0.093 | 1370 | 45 | min |
| 15 | 70% | ‡ | 0.093 | 0.11 | 1360 | 45 | min |
| 16 | 60% | ** | 0.1 | 0.09 | 230 | 3.5 | min |
| 17 | 60% | ** | 0.53 | 0.62 | 4310 | 4.5 | min |
| 18 | 60% | ** | 0.265 | 0.32 | 1070 | 4 | min |
| 19 | 60% | ** | 0.4 | 0.47 | 1270 | 7 | min |
| 20 | 52% | ** | 0.4 | 0.47 | 3350 | 3.5 | min |

One of each of three different methods was used to constitute each of the samples decribed in Table 7, above. Those samples designated with a “†” in the “Method of Prep” column of Table 7 were prepared by first making batches of the vehicle containing no viscosity enhancing substances. Different levels of xanthum gum and Avicel RC-591 were then added, and the resulting mixture shaken, using a mechanical shaker, until dissolution was achieved. Those samples designated with a “‡” were constitued using a propeller mixer, by first adding water to a container followed by viscosity enhancing substances. After dissolution of the viscosity enhancing substances was achieved, the rest of the vehicle excipients were added. Finally, those samples designated with a “**” were prepared by making a powder blend of all the excipients and then adding water by weight to constitute. This last set of samples was shaken by hand.

For all of the 50% solids and 60% solids vehicles, the dry excipient components were added to the container in the order listed in Table 7, except as noted above. An appropriate amount of water was added by weight to the bottle and shaken vigorously by hand until all components were in solution. Constitution time was recorded.

In the 60% solids vehicles, the levels of sodium chloride, citric acid, sodium citrate and sodium benzoate were also increased in order to achieve the same levels of those excipients as in the control oral suspension vehicle. In addition, fructose was removed from the formulation and the total amount of sugars was reduced to 60% solids. The amount of sugars was decreased from 70% to 60% in order to dissolve more xanthan gum and Avicel RC-591 and to achieve the target viscosity of 2500-4000 cps. Additional adjustments were made to the oral suspension formulation after the method for the preparation of the vials for release rate studies had been evaluated. The amount of sugar solids was further reduced from 60% to 50% (Table 7) to reduce constitution time.

The initial target dissolution time in this study was 3-4 minutes and the initial target viscosity was 2500-4000 cps. Using the 70% solids formulation and adding xanthan gum varying in range from 0.12-0.57 w/w % and Avicel® RC-591 varying in range from 0.14-0.68 w/w % resulted in a wide range of viscosities. Vehicle 6 had a viscosity that fell closest to the target viscosity, at 2460 cps. However, the excipients for vehicle 6 took more than 2 hours to dissolve.

### Example 8

### Testing Methods of Suspension Vehicle Preparation

A new method of vehicle preparation was also explored (see vehicles 9-15 of Table 7), in order to dissolve the viscosity enhancing substances prior to the addition of the other excipients. It had been observed in previous vehicle preparations that the other excipients dissolved before the xanthan gum and Avicel. In this set of experiments, xanthan gum ranged from 0.005 to 0.093 w/w % and Avicel® RC-591 ranged from 0.007 to 0.11 w/w %. A viscosity of 1370 cps (vehicle 14) was obtained, however even with this lower viscosity a mixing time of 45 minutes was needed to completely dissolve the excipients. From these studies, it became clear that it would be difficult to prepare a 70% solids vehicle with a viscosity of 2500-4000 cps that could also be constituted in a reasonable amount of time. Therefore additional vehicles containing lower solids content were examined.

The next several vehicles (vehicle 16-19 in Table 7) were prepared with 60% by weight sugars with varying levels of viscosity enhancing substances, and were all constituted by hand. For these vehicles, xanthan gum ranged from 0.1 to 0.53 w/w % and Avicel® RC-91 ranged from 0.09 to 0.62 w/w %. A viscosity of 4310 cps was measured for vehicle 17 and contained 0.53% xanthan gum and 0.62% Avicel® RC-591. The constitution time for this sample was 4.5 minutes, close to the target dissolution time of 3-4 minutes. Additional modifications to the vehicle formulation resulted in an improved 50% vehicle which contained 0.4% xanthan gum and 0.47% Avicel® RC-591. The viscosity of this 50% solids vehicle was 3350 cps and its dissolution time was approximately 3.5 minutes. This is a practical shaking time in a pharmacy setting.

Finally, a linexolid release rate study was conducted on Microcap particles suspended in control, 70% solids, and 50% solids vehicles, after constitution by hand shaking, as described immediately above. The results of the study are illustrated in Table 8, below. A plot of the data in Table 8 can be found in FIG. 5, below.

TABLE 8

| Sample | | | | | Average |
|---|---|---|---|---|---|
| Number | | | Linezolid | Linezolid | Linezolid |
| (Notebook | | | (mg/ml) | (mg/ml) | Conc. |
| Reference) | Vehicle | Day | Vial 1 | Vial 2 | mg/g |
| 1 | Control | 1 | 0.204 | 0.206 | 0.205 |
| | | 3 | 0.316 | 0.327 | 0.322 |
| | | 7 | 0.561 | none detected | 0.561 |
| | | 10 | 0.679 | 0.697 | 0.688 |
| 2 | Control | 2 | 0.21 | 0.22 | 0.215 |
| | | 4 | 0.28 | 0.27 | 0.275 |
| | | 7 | 0.40 | 0.43 | 0.42 |
| | | 10 | 0.58 | 0.50 | 0.54 |
| 3 | 70% solids | 1 | 0 | 0 | 0.00 |
| | | 3 | 0.005 | 0.005 | 0.005 |
| | | 7 | 0.010 | 0.011 | 0.010 |
| | | 10 | 0.014 | 0.014 | 0.014 |
| 4 | 50% solids | 1 | 0.026 | none detected | 0.026 |
| | | 3 | 0.076 | 0.099 | 0.088 |
| | | 7 | 0.096 | 0.097 | 0.097 |
| | | 10 | 0.125 | 0.161 | 0.143 |
| 5 | 50% solids | 2 | 0.036 | 0.037 | 0.036 |
| | | 4 | 0.053 | 0.056 | 0.054 |
| | | 7 | 0.078 | 0.076 | 0.077 |
| | | 10 | 0.100 | 0.101 | 0.100 |

Of the samples tested in this example, the coated linezolid particles released the least amount of linezolid into the vehicle with a 70% solids content. However, as noted above, the constitution time for that particular vehicle is too slow to make its use practical for pharmaceutical applications. The coated linezolid particles released the greatest amount of linezolid into the control vehicles. The coated linezolid particles released dignificantly less linezolid into the vehicles with a 50% solids content than into the control vehicles. As noted above, this last vehicle also has a significantly lower constitution time than the vehicle with a 70% solids content. Together, the low constitution time and low mass transport of linezolid into this last type of vehicle make it a very practical formulation for use in constitutable suspensions of coated linezolid particles, such as the Microcap particles suspended in the vehicles tested in this Example.

We claim:

1. A dry formulation comprising:
at least one dose of coated oxazolidinone particles, each of the particles comprising a core comprising an oxazolidinone, and a polymer film coating at least part of the core; and
a mixture of sugars, comprising sorbitol and at least one other sugar, the sorbitol being present in a non-diarrheogenic amount per dose of the coated oxazolidinone particles.

2. The dry formulation of claim 1, wherein the oxazolidinone is selected from the group consisting of linezolid, N-((5S)-3-(3-fluoro-4-(4-(2-fluoroethyl)-3-oxopiperazin-1-yl)phenyl)-2-oxooxazolidin-5-ylmethyl)acetamide, (S)-N-[[3-[5-(3-pyridyl)thiophen-2-yl]-2-oxo-5-oxazolidinyl]methyl]acetamide, (S)-N-[[3-[5-(4-pyridyl)pyrid-2-yl]-2-oxo-5-oxazolidinyl]methyl]acetamide hydrochloride and N-[[(5S)-3-[4-(1,1-dioxido-4-thiomorpholinyl)-3,5-difluorophenyl]-2-oxo-5-oxazolidinyl]methyl]acetamide.

3. The dry formulation of claim 1, wherein the oxazolidinone is linezolid.

4. The dry formulation of claim 1, the polymer film coating at least 80% of the core of each of the particles.

5. The dry formulation of claim 1, wherein the coated oxazolidinone particles are microencapsulated oxazolidinone particles.

6. The dry formulation of claim 1, wherein the coated oxazolidinone particles are coacervated oxazolidinone particles.

7. The dry formulation of claim 1, wherein the polymer film comprises a hydrophobic polymer selected from the group consisting of: vinyl acetate, vinyl chloride, vinyl carbonate, methacrylic acid, a polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate.

8. The dry formulation of claim 1, wherein the polymer film comprises methacrylic acid or a polymethacrylic acid copolymer.

9. The dry formulation of claim 1, wherein the polymer film is ethylcellulose.

10. The dry formulation of claim 1, wherein the polymer film further comprises a surfactant.

11. The dry formulation of claim 1, wherein the coated oxazolidinone particles have an average particle size of about 50 μm to about 600 μm.

12. The dry formulation of claim 1, the mixture of sugars being about 40% to about 90% by weight of the formulation.

13. The dry formulation of claim 1, the mixture of sugars being about 45% to about 55% by weight of the formulation.

14. The dry formulation of claim 1, wherein the weight ratio of sorbitol to the at least one other sugar in the dry formulation is at least about 1.3:1.

15. The dry formulation of claim 1, wherein the at least one other sugar is selected from the group consisting of: a polysaccharide, a oligosaccharide, a disaccharide, and a monosaccharide, or a mixture of two or more sugars selected from one or more of the above.

16. The dry formulation of claim 1, wherein the at least one other sugar comprises sucrose.

17. The dry formulation of claim 16, the mixture of sugars comprising about 20% to about 35% by weight sucrose, and about 25% to about 40% by weight sorbitol.

18. The dry formulation of claim 16, wherein the at least one other sugar further comprises fructose.

19. The dry formulation of claim 18, the mixture of sugars comprising about 30% to about 40% by weight sucrose, about 30% to about 40% by weight sorbitol, and about 5% to about 15% by weight fructose.

20. The dry formulation of claim 1, further comprising a flavoring agent.

21. The dry formulation of claim 1, further comprising a viscosity enhancing substance.

22. The dry formulation of claim 21, wherein the viscosity enhancing substance is selected from the group consisting of an alginate, carageenin, agar-agar, tragacanth gum, xanthan gum, guar gum, caroba gum, karaya gum, modified corn starch, carboxymethyl cellulose, and crystalline cellulose alone or in combination with other hydrocolloids.

23. The dry formulation of claim 21, wherein the viscosity enhancing substance is a mixture of xanthan gum, microcrystalline cellulose, and sodium carboxymethylcellulose.

24. The dry formulation of claim 21, wherein the viscosity enhancing substance facilitates suspension of the particles in an aqueous solution in less than about ten (10) minutes after addition of the aqueous solution to the dry formulation.

25. The dry formulation of claim 21, wherein the viscosity enhancing substance facilitates suspension of the particles in an aqueous solution in less than about three (3) minutes after addition of the aqueous solution to the dry formulation.

26. The dry formulation of claim 1, wherein the aqueous liquid is water.

27. A suspension comprising a dry formulation suspended in an aqueous solution, the dry formulation comprising:
at least one dose of coated oxazolidinone particles, each of the particles comprising a core comprising an oxazolidinone, and a polymer film coating at least part of the core; and
a mixture of sugars comprising sorbitol and at least one other sugar, the sorbitol being present in a non-diarrheogenic amount per dose of the coated oxazolidinone particles.

28. The suspension of claim 27, wherein the oxazolidinone is selected from the group consisting of linezolid, N-((5S)-3-(3-fluoro-4-(4-(2-fluoroethyl)-3-oxopiperazin-1-yl)phenyl)-2-oxooxazolidin-5-ylmethyl)acetamide, (S)-N-[[3-[5-(3-pyridyl)thiophen-2-yl]-2-oxo-5-oxazolidinyl]methyl]acetamide, (S)-N-[[3-[5-(4-pyridyl)pyrid-2-yl]-2-oxo-5-oxazolidinyl]methyl]acetamide hydrochloride and N-[[(5S)-3-[4-(1,1-dioxido-4-thiomorpholinyl)-3,5-difluorophenyl]-2-oxo-5-oxazolidinyl]methyl]acetamide.

29. The suspension of claim 27, wherein the oxazolidinone is linezolid.

30. The suspension of claim 27, the polymer film coating at least 80% of the core of each of the particles.

31. The suspension of claim 27, wherein the coated oxazolidinone particles are microencapsulated oxazolidinone particles.

32. The suspension of claim 27, wherein the coated oxazolidinone particles are coacervated oxazolidinone particles.

33. The suspension of claim 27, wherein the polymer film comprises a hydrophobic polymer selected from the group consisting of: vinyl acetate, vinyl chloride, vinyl carbonate, methacrylic acid, a polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate.

34. The suspension of claim 27, wherein the polymer film comprises methacrylic acid or a polymethacrylic acid copolymer.

35. The suspension of claim 27, wherein the polymer film is ethylcellulose.

36. The suspension of claim 27, wherein the polymer film further comprises a surfactant.

37. The suspension of claim 27, wherein the coated oxazolidinone particles have an average particle size of about 50 μm to about 600 μm.

38. The suspension of claim 27, the mixture of sugars being about 40% to about 90% by weight of the formulation.

39. The dry formulation of claim 27, the mixture of sugars being about 45% to about 55% by weight of the formulation.

40. The suspension of claim 27, wherein the weight ratio of sorbitol to the at least one other sugar in the dry formulation is at least about 1.3:1.

41. The suspension of claim 27, wherein the at least one other sugar is selected from the group consisting of: a polysaccharide, a oligosaccharide, a disaccharide, and a monosaccharide, or a mixture of two or more sugars selected from one or more of the above.

42. The suspension of claim 27, wherein the at least one other sugar comprises sucrose.

43. The suspension of claim 42, the mixture of sugars comprising about 20% to about 35% by weight sucrose, and about 25% to about 40% by weight sorbitol.

44. The suspension of claim 42, wherein the at least one other sugar further comprises fructose.

45. The suspension of claim 44, the mixture of sugars comprising about 30% to about 40% by weight sucrose, about 30% to about 40% by weight sorbitol, and about 5% to about 15% by weight fructose.

46. The suspension of claim 27, further comprising a flavoring agent.

47. A method of treating or preventing a gram-positive bacterial infection comprising:
orally administering to a subject a therapeutically effective dose of a suspension formulation, comprising:
coated linezolid particles, each of the particles comprising a core comprising linezolid and a polymer film coating at least part of the core; and
a mixture of sugars comprising sorbitol and at least one other sugar, the sorbitol being present in a non-diarrheogenic amount.

48. The method of claim 47, wherein the subject is a mammal.

49. The method of claim 47, wherein the subject is a human being.

50. The method of claim 47, wherein the gram-positive bacterial infection treated or prevented is due to a bacteria of a genera selected from the group consisting of: Staphylococcus (e.g., Staphylococcus aureus, Staphylococcus epidermidis), Streptococcus (e.g., Streptococcus viridans, Streptococcus pneumoniae), Enterococcus, Bacillus, Corynebacterium, Chlamydia and Neisseria.

51. The method of claim 47, the polymer film coating at least 80% of the core of each of the particles.

52. The method of claim 47, wherein the coated oxazolidinone particles are microencapsulated oxazolidinone particles.

53. The method of claim 47, wherein the coated oxazolidinone particles are coacervated oxazolidinone particles.

54. The method of claim 47, wherein the polymer film comprises a hydrophobic polymer selected from the group consisting of: vinyl acetate, vinyl chloride, vinyl carbonate, methacrylic acid, a polymethacrylic acid copolymer, other polymethylmethacrylates, ethyl cellulose, nitrocellulose, vinylidene chloride-acrylonitrile copolymer, acrylonitrile-styrene copolymer, polyethylene, polyethylene oxide, polystyrene, ethylene vinyl acetate, cellulose acetate, cellulose acetate phthalate, cellulose acetate butyrate, hydroxypropylmethylcellulose phthalate.

55. The method of claim 47, wherein the polymer film comprises methacrylic acid or a polymethacrylic acid copolymer.

56. The method of claim 47, wherein the polymer film is ethylcellulose.

57. The method of claim 47, wherein the polymer film further comprises a surfactant.

58. The method of claim 47, wherein the coated oxazolidinone particles have an average particle size of about 50 μm to about 600 μm.

59. The method of claim 47, the mixture of sugars being about 40% to about 90% by weight of the formulation.

60. The dry formulation of claim 47, the mixture of sugars being about 45% to about 55% by weight of the formulation.

61. The method of claim 47, wherein the weight ratio of sorbitol to the at least one other sugar in the dry formulation is at least about 1.3:1.

62. The method of claim 47, wherein the at least one other sugar is selected from the group consisting of: a polysaccharide, a oligosaccharide, a disaccharide, and a monosaccharide, or a mixture of two or more sugars selected from one or more of the above.

63. The method of claim 47, wherein the at least one other sugar comprises sucrose.

64. The method of claim 63, the mixture of sugars comprising about 20% to about 35% by weight sucrose, and about 25% to about 40% by weight sorbitol.

65. The method of claim 63, wherein the at least one other sugar further comprises fructose.

66. The method of claim 65, the mixture of sugars comprising about 30% to about 40% by weight sucrose, about 30% to about 40% by weight sorbitol, and about 5% to about 15% by weight fructose.

67. The method of claim 47, further comprising a flavoring agent.
